(19) United States
(12) Patent Application Publication
(10) Pub. No.: US 2010/0316990 A1
(43) Pub. Date: Dec. 16, 2010

(54) Biomarkers for HPV-Induced Cancer

(75) Inventors: William Dynan, Martinez, GA (US); Hilal Arnouk, Birmingham, AL (US); Mark Merkley, Augusta, GA (US); Jeffrey Lee, Martinez, GA (US); Daron Ferris, Evans, GA (US); Hubert Stoppler, Foster City, CA (US); Robert H. Podolsky, Martinez, GA (US)

Correspondence Address: Pabst Patent Group LLP, 1545 PEACHTREE STREET NE, SUITE 320, ATLANTA, GA, 30309, US

(73) Assignee: Medical College of Georgia Research Institute, Inc Georgia

(21) Appl. No.: 12/745,404

(22) PCT Filed: Jan. 16, 2009
(86) PCT No.: PCT/US09/31302

§ 371 Date: May 28, 2010

(30) Foreign Application Priority Data
Jan. 16, 2008 (US) 61011181

(51) Int. Cl. C12Q 1/70; C12Q 1/02; C12Q 1/26

(52) U.S. Cl. 435/5; 435/29; 435/25

(57) ABSTRACT

Biomarkers that correlate with progression to neoplasia in human papillomavirus (HPV) induced cancer, for example cervical cancer have been identified. These biomarkers can be used to diagnosis or assist in the diagnosis of HPV-induced cancer. They can also be used to increase the positive predictive value of current screening modalities. In addition, they can provide insights into the biology of HPV-induced cancer and thus provide leads for the development of nonsurgical therapies. Exemplary biomarkers include cornulin, PA28 β, DJ-1, actin, transthyretin, HSPB1, CV intracellular channel 1, cytokeratin 8, transferrin, Hsρβ6 (HSP20), aflatoxin reductase, α2 type I collagen, creatine kinase B, cytokeratin 13 GST π, PA28 α, Manganese SOD, lamin A/C, serpin B1 (elastase inhibitor), serpin B3 (SCAA1), cytokeratin 10, cytokeratin 6A, and trp-tRNA synthetase. Preferred biomarkers for HPV-induced cancer include cornulin, DJ-1, PA28 α, and PA28 β, trp-tRNA synthetase, HSPβ6, creatine kinase B, aflatoxin reductase, GST π, transthyretin, transferrin, α2-type 1 collagen, and combinations thereof.

### CROSS REFERENCE TO RELATED APPLICATIONS

This application claims benefit of and priority to U.S. Provisional Patent Application No. 61/011,181 filed on Jan. 16, 2008, and where permissible is incorporated by reference in its entirety.

### FIELD OF THE INVENTION

The invention is generally related to the field of oncology, in particular, to biomarkers for cervical cancer.

### BACKGROUND OF THE INVENTION

Cancer of the uterine cervix is a significant cause of mortality, responsible for about 200,000 deaths per year among women worldwide [1]. Screening for early detection, using the Papanicolaou (Pap) test, has reduced mortality about four-fold in developed countries [2, 3]. Exfoliated cervical cells are evaluated based on alterations in nuclear and cellular morphology using the Bethesda classification system [4].

Despite its success in reducing mortality, the Pap test has shortcomings. Abnormal or ambiguous findings, which occur in about 3 million of the 55 million Pap smears performed annually in the US, necessitate costly and sometimes invasive follow-up. The accuracy of the Pap test has been studied extensively, and meta-analysis indicates that high specificity and sensitivity cannot be achieved concurrently [5]. Classification of both Pap smears and follow-up biopsies is subject to high inter-observer variability, with agreement on grading of biopsy specimens only 40% to 80% more than expected by chance alone [6]. In addition, the natural history of cervical premalignant lesions shows great individual variability. Some 40-70% of low grade lesions will regress without treatment, whereas smaller percentages will progress to a higher-grade lesion or to invasive cancer [7]. The decision to surgically ablate low-grade lesions is particularly problematic, as only one to two women per 1000 progress to invasive carcinoma within 24 months, and the procedure itself carries risk [8, 9]. Molecular markers to distinguish individual patients with a high risk of progression would clearly be valuable. Such markers might also be therapeutic targets, expanding the options for non-surgical treatment.

One approach that has been explored for improving the accuracy of cervical cancer screening is to test for the presence of high-risk type human papillomavirus (HPV) DNA following an ambiguous Pap test result. The rationale is that high-risk type HPV is the initiating agent in virtually all cervical carcinomas (reviewed in refs. [10, 11]). In patients with ambiguous Pap test results, HPV DNA assays have been shown to be preferable to repeat cytology [12]. Surrogate protein markers for HPV infection have also been used, including high-level expression of the cyclin-dependent kinase inhibitor, p16(Ink4a), and the expression of a marker of cell proliferation, Ki-67, in normally non-dividing cells of the upper layers of the epithelium [13-15]. A limitation in using HPV or surrogate markers for diagnosis is that infection with high-risk type HPV is relatively common (point prevalence=3.4% [16]) and many infections clear spontaneously. It would be useful to have a test to detect the transition from infected cells, which proliferate simply in response to viral oncoprotein expression, and virally transformed cells, which have accumulated additional genetic and epigenetic changes during a latency period. There are currently no clinically useful molecular markers for detecting this transition.

Therefore it is an object of the invention to provide biomarkers for detecting the transition of virally infected cells to virally transformed cells in HPV-induced cancer.

It is another object to provide methods for distinguishing between virally infected and virally transformed cells in HPV-induced cancer.

It is another object to provide methods for diagnosing or assisting in the diagnosis of HPV-induced cancer.

### SUMMARY OF THE INVENTION

Biomarkers that correlate with progression to neoplasia in human papillomavirus (HPV) induced cancer, for example cervical cancer have been identified. These biomarkers can be used to diagnosis or assist in the diagnosis of HPV-induced cancer. They can also be used to increase the positive predictive value of current screening modalities. In addition, they can provide insights into the biology of HPV-induced cancer and thus provide leads for the development of nonsurgical therapies. Exemplary biomarkers include cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13 GST π, PA28 α, Manganese SOD, lamin A/C, serpin B1 (elastase inhibitor), serpin B3 (SCAA1), cytokeratin 10, cytokeratin 6A, and trp-tRNA synthetase. Preferred biomarkers for HPV-induced cancer include cornulin, DJ-1, PA28 α, and PA28 β, trp-tRNA synthetase, HSPβ6, creatine kinase B, aflatoxin reductase, GST π, transthyretin, transferrin, α2-type 1 collagen, and combinations thereof.

A preferred embodiment provides a method for detecting the transition of virally infected cells to virally transformed cells in HPV-induced cancer by determining the levels of one or more biomarkers for HPV-induced cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cell or tissue sample and comparing the levels of the one or more biomarkers for HPV-induced cancer in the cell or tissue sample to a control or reference level. Levels of PA28β, DJ-1 protein, actin, Cl⁻ intracellular channel 1, cytokeratin 8, creatine kinase B, PA28 α in the cell or tissue sample that are higher than levels in noncancer cells indicates the presence of virally transformed cells. Levels of cornulin, transthyretin, HSPB1, transferrin, Hspβ6 (HSP20), aflatoxin reductase, α2 type I collagen, cytokeratin 13, GST π, and cytokeratin 10 in the cell or tissue sample that are lower than levels in noncancer cells is indicative of virally transformed cells. Representative HPV-induced cancers include, but are not limited to cancer of the cervix, rectum, larynx, orthopharynx, nasopharynx, mouth, head and neck.

One embodiment provides a method for distinguishing invasive cervical cancer cells, premalignant cervical cells, and noncancer cervical cells by determining the levels of one or more biomarkers for cervical cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cervical cell or cervical tissue sample and comparing the levels of the one or more biomarkers for cervical cancer in the cervical cell or cervical tissue sample to a control or reference level. The method also includes identifying invasive cervical cancer cells, premalignant cervical cells, and noncancer cervical cells based on the levels of the one or more biomarkers detected in the cells. The method optionally includes reporting the presence or absence of invasive cervical cancer cells in the cervical cell or cervical tissue sample. Preferably, the levels of the one or more biomarkers are detected using mass spectometry or immunocytochemistry. When levels of cornulin, transthyretin, HSPB1, transferrin, Hspβ6 (HSP20), aflatoxin reductase, α2 type I collagen, cytokeratin 13, GST π, and cytokeratin 10 in the cell or cervical tissue sample are lower than levels in noncancer cells. invasive cervical cancer cells are indicated. When levels of PA28β, DJ-1 protein, actin, Cl⁻ intracellular channel 1, cytokeratin 8, creatine kinase B, PA28 α in the cell or cervical tissue sample are higher than levels in noncancer cells, invasive cervical cancer cells are indicated.

Another embodiment provides a method for monitoring the effects of a drug in the treatment of HPV-induced cancer by determining the levels of one or more biomarkers for HPV-induced cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cell or tissue sample from subject before and after treatment with the drug and identifying changes in levels of the one or more biomarkers in the cervical cell or cervical tissue sample after treatment with the drug relative to before treatment of the drug wherein the changes in levels of the one or more biomarkers are indicative of effects of the treatment with the drug. Preferably, the cell or tissue sample is infected with HPV.

Still another embodiment provides a method for selecting lead compounds for drug development for the treatment of HPV-induced cancer by contacting a test compound with one or more biomarkers for HPV-induced cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin β1 (elastase inhibitor), serpin B3 (SCAA1), cytokeratin 10, cytokeratin 6A, and trp-tRNA synthetase assaying for binding of the test compound to one or more of the biomarkers. The method also includes selecting the test compound that binds to one or more of the biomarkers for HPV-induced cancer for drug development for the treatment of HPV-induced cancer.

One embodiment provides a method for determining the status of HPV-induced cancer, for example cervical cancer by obtaining a sample of cervical tissue from a subject and quantifying levels of cornulin in the sample of cervical tissue. Decreased levels of cornulin in the sample of cervical tissue relative to levels of cornulin in noncancer cells is indicative of invasive cervical cancer cells.

Still another embodiment provides a method for distinguishing premalignant cells from invasive cancer cells by obtaining a sample of tissue from a subject and quantifying levels of trp-tRNA synthetase in the sample of tissue, wherein elevated levels of trp-tRNA synthetase in the sample of tissue relative to a reference level of trp-tRNA in premalignant cells is indicative of invasive cancer cells.

### BRIEF DESCRIPTION OF THE DRAWINGS

FIG. 1A is a schematic diagram of the experimental design to identify biomarkers that correlate with progression to neoplasia in cervical cancer. FIG. 1B is a schematic diagram of an exemplary analytical workflow to identify biomarkers that correlate with progression to neoplasia in cervical cancer.

FIGS. 2A-D are graphical representations of the abundance of cornulin, PA28 β, HSPB1, and MnSOD respectively in normal, HSIL, and cancer samples. Relative abundance values (Y axis) is expressed on a logarithmic scale, with each unit increment representing a 2-fold change. Each circle indicates an individual tissue sample. Patient-matched samples are connected by the dashed lines.

FIGS. 3A-D are graphical representations of immunological staining of frozen sections from patients in normal, HSIL, and cancer groups stained with A., anti-cornulin, B., anti-PA28β, C., anti-Hsp27 (HSPB1), and D., anti-manganese superoxide dismutase. Each panel represent results of scoring on a standard 0-3 scale.

FIGS. 4A and B are histograms of staining intensity of commercial tissue microarray with samples drawn from an independent patient cohort. Scoring was on a standard 0-3 scale.

FIG. 5A is a histogram showing the coefficient of variation (percent) versus number of spots for normal cervix and cancer for total features. FIG. 5B is a histogram showing the coefficient of variation (percent) versus number of spots for normal cervix and cancer for differentially expressed features.

FIG. 6 Graphs display the relative abundance of 31 proteins that were selected for mass spectrometry analysis. Relative abundance values (Y axis) are expressed on a logarithmic scale, with each unit increment representing a 2-fold change. Each circle indicates an individual tissue sample. Patient-matched samples are connected by dashed lines. Brackets indicate intergroup comparisons that met the criteria for candidate biomarkers

### DETAILED DESCRIPTION OF THE INVENTION

### I. Definitions

The term “biomarker” refers to an organic biomolecule which is differentially present in a sample taken from a subject of one phenotypic status (e.g., having a disease) as compared with another phenotypic status (e.g., not having the disease) or uninvolved normal tissue from the same individual. A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity. Preferred biomarkers are proteins.

The terms “individual”, “host”, “subject”, and “patient” are used interchangeably herein, and refer to a mammal, including, but not limited to, humans, rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

The term “drug” refers to small molecules, protein therapeutics, vaccines, and immunomodulators.

### II. Biomarkers for HPV Induced Cancer

Biomarkers that correlate with progression to neoplasia in HPV-induced cancer have been identified. These biomarkers can be used to diagnosis or assist in the diagnosis of HPV-induced cancers such as cancer of the cervix, rectum, larynx, orthopharynx, nasopharynx, mouth, head and neck. They can also be used to increase the positive predictive value of current screening modalities. In addition, they can provide insights into the biology of HPV-induced cancer and thus provide leads for the development of nonsurgical therapies. To identify biomarkers, proteomic patterns were analyzed from samples representing normal, premalignant, and cancer tissue. A dedicated patient sample collection system, LCM to separate lesional tissue from surrounding normal tissue, and a sensitive analytical methodology were used to allow profiling with only a few micrograms of protein. It is believed that this is the first study to simultaneously compare normal cervical tissue, cervical intraepithelial neoplasia, and invasive cervical cancer tissue using the same proteomic methodology.

There were significant changes in expression of many proteins, of which 23 have been identified at the molecular level (Table 1). These biomarkers include cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13 GST π, PA28 α, Manganese SOD, lamin A/C, serpin β1 (elastase inhibitor), serpin B3 (SCAA1), cytokeratin 10, cytokeratin 6A, and trp-tRNA synthetase, Twelve have been seen before in HSIL or cancer, and results are generally concordant with the prior literature. Eleven proteins are that have not previously been linked to HPV-induced cancers, for example cervical cancer include cornulin, DJ-1, PA28 α, and PA28 β, trp-tRNA synthetase, HSPβ6, creatine kinase B, aflatoxin reductase, GST π, transthyretin, transferrin, and α2-type 1 collagen. Initial (technical) validation was performed by randomly sampling a small number of specimens from the original cohort. Results agreed with the 2D-DIGE (Difference Gel Electrophoresis) analysis, lending confidence in the technical quality of the 2D-DIGE and mass spectroscopy (MS) data. Extensive immunohistochemistry studies for two proteins of particular interest, cornulin and HSPB1, which drew on a different patient cohort with larger numbers of specimens and additional disease states were also performed.

The results provided herein emphasize the power of using matched patient samples. In the 2D-DIGE experiments several proteins were identified where there was a significant change in expression between individual normal-HSIL pairs, even though the range of expression values for the normal and HSIL groups as a whole overlapped. These pairings were preserved in the technical validation study using the frozen sections. In the tissue microarrays however, samples were not patient-matched. Although this makes the tissue microarray somewhat less powerful, results extended the initial 2D-DIGE findings.

In principle, there are at least three processes that have the potential to change the proteomic profile during cervical cancer progression: (1) effects resulting from direct interaction of HPV oncoproteins with cellular proteins, (2) stochastic effects resulting from the combination of cell proliferation, genomic instability, and selective pressure during the latency period that is required for development of HSIL and cancer, and (3) emergent properties resulting from interactions of lesional tissue with the tissue microenvironment. Patterns ascribable to all three processes appear to be present in the proteomic data.

A. Source of the Biomarkers

The disclosed biomarkers for cervical cancer are biomolecules, preferably proteins. One embodiment provides these biomolecules in isolated form. The preferred biological source for detection of the biomarkers is cervical tissue including biopsy material from the cervix. However, in other embodiments, the biomarkers can be isolated from biological fluids, cervical secretions, exfoliated cervical cells, cervical tissue, HPV-infected cells or HPV-infected tissue, urine, blood, and serum. The biomarkers can be isolated by any method known in the art, based on both their mass and their binding characteristics. For example, a sample containing the biomolecules can be subject to laser capture microdissection (LCM) and 2D-difference gel electrophoresis as described herein. Other isolation techniques include chromatographic fractionation subject to further separation by, e.g., acrylamide gel electrophoresis. Knowledge of the identity of the biomarker also allows their isolation by immunoaffinity chromatography.

B. Methods of Detecting Biomarkers

The disclosed biomarkers for cervical cancer can be detected by any suitable method. Detection paradigms that can be employed include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally include solid substrates and have a generally planar surface, to which a capture reagent (also called an adsorbent or affinity reagent) is attached. Frequently, the surface of a biochip includes a plurality of addressable locations, each of which has the capture reagent bound there.

Protein biochips are biochips adapted for the capture of polypeptides. Many protein biochips are described in the art. These include; for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden).

1. Detection by Mass Spectrometry

In a preferred embodiment, the biomarkers are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

a. MALDI

In a preferred mass spectrometry method, following trypsin digestion, extracted peptides are spotted onto a 192-well MALDI-TOF target plate for the Applied Biosystems Incorporated (ABI) 4700 Proteomics Analyzer. Automated MALDI-TOF mass spectrometry provides a peptide mass fingerprint. In addition, peptides (excluding trypsin peaks) can be subjected to collision-induced dissociation to obtain sequence information. Spectra are searched using the GPS Explorer (ABI) search tool and Mascot algorithm (Matrix Biosciences) against the NCBInr protein database.

In general, the biomarkers can be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

b. SELDI

Another mass spectrometric technique for detecting the disclosed biomarkers is “Surface Enhanced Laser Description and Ionization” or “SELDI.”. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

Biospecific adsorbent” refers to an adsorbent including a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. A “bioselective adsorbent” refers to an adsorbent that binds to an analyte with an affinity of at least 10⁻⁸ M.

Protein biochips produced by Ciphergen Biosystems, Inc. include surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with carboimidizole, expoxide) and PG-20 (protein G coupled through carboimidizole). Hydrophobic ProteinChip arrays have isopropyl or nonylphenoxy-poly(ethylene glycol)methacrylaate functionalities. Anion exchange ProteinChip arrays have quaternary ammonium functionalities. Cation exchange ProteinChip arrays have carboxylate functionalities. Immobilized metal chelate ProteinChip arrays have have nitriloacetic acid functionalities that adsorb transition metal ions, such as copper, nickel, zinc, and gallium, by chelation. Preactivated ProteinChip arrays have carboimidizole or epoxide functional groups that can react with groups on proteins for covalent binding.

In general, a probe with an adsorbent surface is contacted with the sample for a period of time sufficient to allow biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties, an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxyhenzoic acid, ferulic acid, and hydroxyaceto-phenone derivatives. In certain embodiments, the energy absorbing molecule is incorporated into a linear or cross-linked polymer, e.g., a polymethaerylate. For example, the composition can be a co-polymer of .alpha.-cyano-4-methacryloyloxycinnamic acid and acrylate. In another embodiment, the composition is a co-polymer of .alpha.-cyano-4-methacryloyloxycinnamic acid, acrylate and 3-(tri-ethoxy)silyl propyl methacrylate. In another embodiment, the composition is a co-polymer of .alpha.-cyano-4-methacryloyloxycinnamic acid and octadecylmethacrylate (“C18 SEND”).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. SEPAR and other forms of SELDI are readily adapted to detecting a biomarker or biomarker profile, pursuant to the present invention.

In an exemplary protocol for the detection of the biomarkers for cervical cancer the biological sample to be tested, e.g., cervical tissue, serum or urine, preferably is subject to pre-fractionation before SELDI analysis. This simplifies the sample and improves sensitivity. A preferred method of pre-fractionation involves contacting the sample with an anion exchange chromatographic material, such as Q HyperD (BioSepra, SA). The bound materials are then subject to stepwise pH elution using buffers at pH 9, pH 7, pH 5 and pH 4. Various fractions containing the biomarker are collected.

The sample to be tested (preferably pre-fractionated) is then contacted with an affinity capture probe comprising an cation exchange adsorbent (preferably a WCX ProteinChip array (Ciphergen Biosystems, Inc.)) or an IMAC adsorbent (preferably an IMAC3 ProteinChip array (Ciphergen Biosystems, Inc.)). The probe is washed with a buffer that will retain the biomarker while washing away unbound molecules. The biomarkers are detected by laser desorption/ionization mass spectrometry.

Alternatively, if antibodies that recognize the biomarker are available, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

c. Other Techniques

Mass spectrometry-based Multi-Reaction Monitoring can also be used to detect the biomarkers. Mass spectrometry-based Multi-Reaction Monitoring has been described by Gerber et al., Proc Natl Acad Sci. USA., 100(12):6940-5 (2003). The strategy has two stages. The first involves identification of suitable tryptic peptides from a candidate biomarker. The process of identifying suitable peptides was discussed and illustrated in the Introduction (as a response to a specific reviewer concern). Standard peptides are then synthesized, corresponding to two or more of these peptides. During synthesis, a stable isotope (e.g. 13C) is inserted at a single amino acid residue. The synthetic peptide is then used as standard for absolute quantification of protein present in a clinical sample. Proteins are extracted from the clinical sample, digested with trypsin, and subjected to mass spectrometry in an ABI 4000 Q Trap system mass spectrometer set to detect the peptide of interest together with the non-isobaric standard peptide. The ratio of normal (sample derived) and heavy (synthetic) peptide will be monitored. An advantage to the method is that the sample can be spiked with many peptides simultaneously, permitting multiple monitoring of different species.

d. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (N/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

2. Detection by Immunoassay

In another embodiment, the biomarkers can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

Traditional immunoassays including, for example, sandwich immunoassays including ELISA or fluorescence-based immunoassays, as well as other enzyme immunoassays can be used for detecting the biomarkers. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

Quantitative immunochemical techniques can also be used. For example the Quantitative Tissue Biomarker Platform from HistoRx can be used to quantified levels of biomarkers. This platform is commercially available and quantitates protein expression within subcellular compartments in tissue sections automatically, with a high level of precision.

C. Specific Biomarkers for HPV-Induced Cancer

1. Biomarkers that Potentially Arise from Direct Interactions of HPV Oncoproteins with Cellular Proteins

HPV E6 and E7 bind directly to p53, Rb, and a number of other cellular proteins (reviewed in references [10, 11]). Effects potentially attributable to direct interactions of HPV oncoproteins with these and other cellular proteins account for at least a quarter of the changes in the data provided in the Examples. Serpin B1, a member of a large family of serine protease inhibitors, binds directly to E7 in a pulldown assay [32]. It is down-regulated in vitro in E7-transfected cells [33], consistent with the down-regulation observed here in HSIL. Glutathione-S-transferase similarly decreases in E7-transfected cells, although it is unknown if this reflects a direct protein-protein interaction [37].

Three other proteins identified herein as biomarkers for HPV-induced cancer are known products of p53 target genes. Creatine kinase B and tryptophanyl tRNA synthetase are p53-repressible enzymes [34, 35] that increased significantly in cancer. Although expression of these proteins may be influenced by factors in addition to p53, the direction of the changes in expression, in both cases, is consistent with HPV E6-mediated loss of p53 function.

Other identified proteins may be regulated indirectly as a result of compromised Rb function in E7-expressing cells, which fosters continued proliferation of cells in the upper layers of squamous epithelium, reducing or blocking terminal differentiation and cornification. The differentiation marker, cornulin, declines in HSIL and further declines in cancer. Cornulin is a member of the “fused gene” family, binds calcium, and is up-regulated in response to deoxycholate-induced stress [36, 37]. It is normally expressed late during epidermal differentiation, but its function is otherwise unknown, and it has not previously been described as a cervical cancer marker. This, taken with the result of the immunohistochemistry experiments, indicate that cornulin is a useful as a diagnostic marker of disease state.

Changes in cytokeratin expression can also be ascribed to loss of the differentiated state. Expression of three cytokeratins (6A, 10, and 13) decreased in HSIL and cancer relative to normal tissue. These three proteins are known markers of keratinocyte differentiation, and the decline is consistent both with loss of the differentiated state and with previous studies of cervical cancer [15]. Cytokeratin 8 was increased in cancer relative to normal tissue, again consistent with previous work [38].

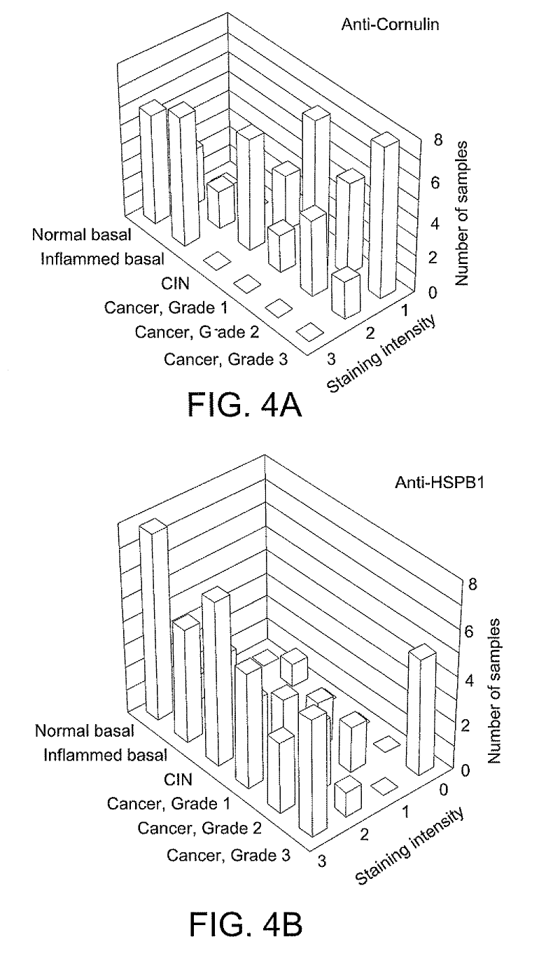

HSPB1 apparently falls into the same category of differentiation markers. The observed decline in cancer specimens was paradoxical, in that expression of this and other HSPs have been widely observed to increase in proteomic studies of cancer cells. Although there are conflicting prior reports about expression in HPV-induced lesions [14, 31, 39], it is believed that HSPB1 may have a specialized function as a cornification chaperone, and it is expressed at high levels in the upper levels of normal stratified epithelium and in in vitro differentiated keratinocytes (FIG. 4 and references [29, 39]). Relatively high levels were seen in normal cervix, a slight decline in HSIL, and a marked decline in cancer, especially in some specimens. A decline in expression in less-differentiated lesions plausibly reflects their inability to undergo terminal differentiation in the presence of HPV oncoproteins. Consistent with this, in the tissue microarray, the highest frequency of HSPB1-negative specimens was in the least-differentiated (grade 3) tumors. It will be of interest to investigate the mechanism of heterogeneity in highgrade cancers and to determine whether HSPB1 status has independent prognostic or predictive value. This will require a separate study, as clinical outcome data are not available for the subjects used here.

Expression of another small heat shock protein, Hspβ6 (Hsp20) also declined in HSIL and cancer. No examples of HSPs that increased significantly in HSIL or cancer were observed.

D. Markers that are Potentially Selected During the Latency Period

Like other human cancers, cervical cancer typically develops only after a long latency period. Effects attributable to variation and selection for growth advantage are expected to occur stochastically during latency; that is, both the timing and whether a given change occurs at all will vary between patients. The oncoprotein, DJ-1, may fall into this category. DJ-1 significantly increased in cancer versus normal tissue, whereas expression values in HSIL showed considerable dispersion. DJ-1 transforms mouse NIH3T3 cells in vitro and is overexpressed in many cancers including: breast, lung, pancreatic, ovarian, and prostate [40-44]. Mechanistic studies show that DJ-1 is a negative regulator of the tumor suppressor, PTEN [45]. Interestingly, although down-regulation of PTEN expression is a negative prognostic indicator in cervical cancer [46, 47], direct mutation or loss of heterozygosity at the PTEN locus is rare [46].

Overexpression of DJ-1 could provide a mechanism for down-regulation in the absence of direct mutation or loss of the PTEN gene. It is well established that deficiency of DJ-1 (also known as PARK7) sensitizes dopaminergic neurons to stress-mediated apoptosis in hereditary Parkinson's disease(reviewed in reference [48]). Regulation of apoptosis appears to be the common link explaining the role of DJ-1 in these disparate diseases. Interestingly, expression of Serpin B1, another biomarker discovered in this study, has previously been shown to be PTEN dependent [49]. Thus down-regulation of PTEN in HSIL could provide another explanation for the observed down regulation of Serpin B1 (in addition to direct interaction of HPV E7 with Serpin B1).

Several other proteins may fall into the category of proteins that are selected during the latency period. Manganese superoxide dismutase, which increased in HSIL, protects against free radical toxicity. High expression has previously been correlated with poor outcomes in cervical cancer [50]. Serpin B3 (SCCA1) declined in HSIL, and Chloride intracellular channel 1 protein increased in cancer; both results are novel in the context of cervical disease.

E. Markers that are Potentially Influenced by Interaction of Lesions with the Microenvironment

Three IFN-γ inducible proteins were identified as up-regulated in cancer. Unlike IFN-α and IFN-β, which are expressed by many cell types, IFN-γ is expressed only by T cells and NK cells. Thus, expression of IFN-γ-inducible genes in cancers cells is expected to occur only as a consequence of cell-cell interactions within the tissue microenvironment. Two of the IFN-γ-inducible proteins, PA28 α and PA28 β, activate the 20S proteasome complex, which presents antigens via the MHC I pathway. Although the up-regulation of these proteins is novel in cervical cancer, up-regulation of PA 28 α has been described previously in infiltrating ductal breast carcinoma [51]. Another IFN-γ-inducible tryptophanyl protein, tRNA synthetase, has been hypothesized to protect cells from tryptophan starvation following IFN-γ-mediated induction of the catabolic enzyme, indoleamine 2,3 dioxygenase [52].

Other proteins that may fall into the category of changes attributable to host-lesion interactions include the serum transporter, transthyretin, which was decreased in cancer and HSIL. Transthyretin is a negative acute-phase serum protein that decreases in inflammatory conditions including many cancers [53]. Transferrin, another serum transporter that decreased in cancer, has been reported to decrease in ovarian cancer [54]. The decrease in extracellular matrix protein, α2-type I collagen that occurred in HSIL may be an indirect effect of IFN-γ, mediated via stimulation of the IRF-1 transcription factor [55].

### III. Methods of Using Biomarkers for HPV-Induced Cancer

A. Diagnosis

1. Single Markers

The biomarkers can be used in diagnostic tests to assess HPV-induced cancer status in a subject, e.g., to distinguish between normal cells, high-risk premalignant cells, and invasive carcinoma. The phrase “HPV-induced cancer status” includes any distinguishable manifestation of the disease, including non-disease. For example, disease status includes, without limitation, the presence or absence of disease (e.g., cancer v. non-cancer), the risk of developing disease, the stage of the disease (e.g., non-invasive or early-stage cancer v. invasive or metastatic cancer), the progress of disease (e.g., progress of disease or remission of disease over time) and the effectiveness or response to treatment of disease. Based on this status, further procedures may be indicated, including additional diagnostic tests or therapeutic procedures or regimens. Representative HPV-induced cancers include, but are not limited to cancer of the cervix, rectum, larynx, orthopharynx, nasopharynx, mouth, head and neck.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives who test as positive. Negative predictive value is the percentage of actual negatives that test as negative.

The biomarkers of this invention show a statistical difference in different cervical cancer statuses of at least p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at east 98% and about 100%.

Each biomarker listed in Table 1 is differentially expressed in cervical cancer, and, therefore, each is individually useful in aiding in the determination of cervical cancer status. The method involves, first, measuring the selected biomarker in a subject sample using the methods described herein, e.g., capture on a MALDI biochip followed by detection by mass spectrometry and, second, comparing the measurement with a diagnostic amount or cut-off that distinguishes a positive cervical cancer status from a negative cervical cancer status. The diagnostic amount represents a measured amount of a biomarker above which or below which a subject is classified as having a particular cervical cancer status. For example, if the biomarker is up-regulated compared to normal during cervical cancer, then a measured amount above the diagnostic cutoff provides a diagnosis of cervical cancer. Alternatively, if the biomarker is down-regulated during cervical cancer, then a measured amount below the diagnostic cutoff provides a diagnosis of cervical cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different cervical cancer statuses and drawing the cut-off to suit the diagnostician's desired levels of specificity and sensitivity.

In one embodiment, cornulin levels are measured in a sample and compared to a control. A representative control is cervical tissue known to be free of HPV-induced cancer from the same or a different individual. A control can also be a reference standard for example a reference protein in the same sample known not to change levels. Cornulin levels are significantly higher in maturing squamous cells of the normal epithelium compared to non-cancer cells, reduced in non-dysplastic cells in HSIL, and non-detectable in invasive cancer cells. Other biomarkers that have reduced levels in invasive cancer cells relative to noncancer cells or are down-regulated in invasive cancer cells include transthyretin, HSPB1, transferrin, Hspβ6 (HSP20), aflatoxin reductase, α2 type I collagen, cytokeratin 13, GST π, and cytokeratin 10. One or more of the biomarkers with reduced levels in invasive cancer cells relative to noncancer cells can be used to select or identify invasive cancer cells, to distinguish invasive cancer cells from noncancer cells, or to assist in the diagnosis of HPV-induced cancer.

Biomarkers for HPV-induced cancer that have increased levels in invasive cancer cells relative to noncancer cells or are upregulated in invasive cancer cells relative to noncancer cells include PA28β, DJ-1, actin, Cl⁻ intracellular channel 1, cytokeratin 8, creatine kinase B and PA28 α. One or more of the biomarkers with increased levels in invasive cancer cells relative to noncancer cells can be used to select or identify invasive cancer cells, to distinguish invasive cancer cells from noncancer cells, or to assist in the diagnosis of HPV-induced cancer.

Biomarkers for HPV-induced cancer that have decreased levels or are downregulated in premalignant cells relative to noncancer cells include cornulin, transthyretin, cytokeratin 13, lamin A/C, serpin B1 (elastase inhibitor), serpin B3(SCCA1), cytokeratin 10, and cytokeratin 6A. One or more of the biomarkers with reduced levels in premalignant cells relative to noncancer cells can be used to select or identify premalignant cells, to distinguish premalignant cells from noncancer cells, or to assist in identifying the status of HPV-induced cancer.

Biomarkers for HPV-induced cancer that have increase levels or are uregulated in premalignant cells relative to noncancer cells include cytokeratin 8 and Manganese SOD. One or more of the biomarkers with increased levels in invasive premalignant cells relative to noncancer cells can be used to select or identify premalignant cells, to distinguish premalignant cells from noncancer cells, or to assist in identifying the status of HPV-induced cancer.

Biomarkers for HPV-induced cancer that have decreased levels or are downregulated in invasive cancer cells relative to premalignant cells include cornulin, PA28 β, HSPB1, transferrin, and α2 type I collagen. One or more of the biomarkers with reduced levels in invasive cells relative to premalignant cells can be used to select or identify invasive cancer cells, to distinguish premalignant cells from noncancer cells, or to assist in identifying the status of HPV-induced cancer.

trp-tRNA synthetase is upregulated in invasive cancer cells relative to premaligant cells. This biomarker can be used to to distinguish premalignant cells from noncancer cells, or to assist in identifying the status of HPV-induced cancer.

Preferred biomarkers for HPV-induced cancer include cornulin, PA28 α, and PA28 β, trp-tRNA synthetase, HSPβ6, creatine kinase B, aflatoxin reductase, GST π, transthyretin, transferrin, α2-type I collagen, and combinations thereof.

2. Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. Thus, in one embodiment, two or more, three or more, four or more or even five or more of the biomarkers in Table 1 can be detected and used to assess the status of cervical cancer in a subject.

B. Determining Risk of Developing Disease

One embodiment provides methods for determining the risk of developing disease in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level.

C. Determining Stage of Disease

Another embodiment provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage. For example, detection biomarker cornulin can be used to distinguish between early-stage (non-invasive) to invasive cervical cancer.

D. Determining Course (Progression/Remission) of Disease

Still another embodiment provides methods for determining the course of disease in a subject. Disease course refers to changes in disease status over time, including disease progression (worsening) and disease regression (improvement). Over time, the amounts or relative amounts (e.g., the pattern) of the biomarkers changes. This method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

E. Subject Management

In certain embodiments of the methods of qualifying cervical cancer status, the methods further include managing subject treatment based on the status. Such management includes the actions of the physician or clinician subsequent to determining cervical cancer status. For example, if a physician makes a diagnosis of cervical cancer, then a certain regime of treatment, such as prescription or administration of chemotherapy, radiation, immunotherapy might follow. Alternatively, a diagnosis of non-cervical cancer or benign cervical-disease might be followed with further testing to determine a specific disease that might the patient might be suffering from. Also, if the diagnostic test gives an inconclusive result on cervical cancer status, further tests may be required.

One embodiment provides a method for selecting a subject for treatment for cervical cancer by detecting the presence or quantity of one or more biomarkers in Table 1 in a sample from a subject suspected of having cervical cancer, comparing the levels of biomarker in the sample to a predetermined standard, wherein the patient is selected for treatment for cervical cancer if certain biomarkers or levels of biomarkers are detected in the sample.

Additional embodiments relate to the communication of assay results or diagnoses or both to technicians, physicians or patients, for example. In certain embodiments, computers will be used to communicate assay results or diagnoses or both to interested parties, e.g.: physicians and their patients. In some embodiments, the assays will be performed or the assay results analyzed in a country or jurisdiction which differs from the country or jurisdiction to which the results or diagnoses are communicated.

In a preferred embodiment a diagnosis based on the presence or absence in a test subject of any the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. In certain embodiments all or some of the method steps, including the assaying of samples, diagnosing of diseases, and communicating of assay results or diagnoses, may be carried out in diverse (e.g., foreign) jurisdictions.

F. Biomarkers in Screening Assays

The biomarkers can be used to screen for compounds that modulate the expression of the biomarkers in vitro or in vivo, which compounds in turn may be useful in treating or preventing cervical cancer in patients. Compounds suitable for therapeutic testing may be screened initially by identifying compounds which interact with one or more biomarkers listed in Table 1. By way of example, screening might include recombinantly expressing a biomarker listed in Table 1, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table 1, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table I may be measured. One of skill in the art will recognize that the techniques used to measure the activity of a particular biomarker will vary desponding on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers of Table I may also be measured. For example, the self-assembly of a multi-protein complex which includes one of the biomarkers of Table I may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table I may be administered to patients who are suffering from or are at risk of developing cervical cancer or other cancer. For example, the administration of a test compound which increases the activity of a particular biomarker may decrease the risk of cervical cancer in a patient if the activity of the particular biomarker in vivo prevents the accumulation of proteins for cervical cancer. Conversely, the administration of a test compound which decreases the activity of a particular biomarker may decrease the risk of cervical cancer in a patient if the increased activity of the biomarker is responsible, at least in part, for the onset of cervical cancer.

At the clinical level, screening a test compound includes obtaining samples from test subjects before and after the subjects have been exposed to a test compound. The levels in the samples of one or more of the biomarkers listed in Table 1 may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table 1 may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers of Table 1 may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with cervical cancer, test compounds will be screened for their ability to slow or stop the progression of the disease.

G. Assessing the Effectiveness of Treatment or Risk for Developing Cervical Cancer

Methods for determining the course of cervical cancer in a subject are also provided. Disease course refers to changes in disease status over time, including disease progression (worsening) and disease regression (improvement). Over time, the amounts or relative amounts (e.g., the pattern) of the biomarkers changes. For example, biomarkers cornulin, transthyretin, and HSPB1 are decreased in disease. Therefore, the trend of these markers, either increased or decreased over time toward diseased or non-diseased indicates the course of the disease. Accordingly, this method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

In yet another example, the biomarkers can be used in heredity studies to determine if the subject is at risk for developing cervical cancer.

### IV. Kits

An exemplary kit includes a solid substrate having a hydrophobic function, such as a protein biochip (e.g., a Ciphergen H50 ProteinChip array, e.g., ProteinChip array) and a sodium acetate buffer for washing the substrate, as well as instructions providing a protocol to measure the biomarkers on the chip and to use these measurements to diagnose HPV-induced cancer or the progression of HPV-induced cancer.

### Examples

### Example 1

### Collection and Analysis of Proteomic Data

### Materials and Methods

Experimental Design

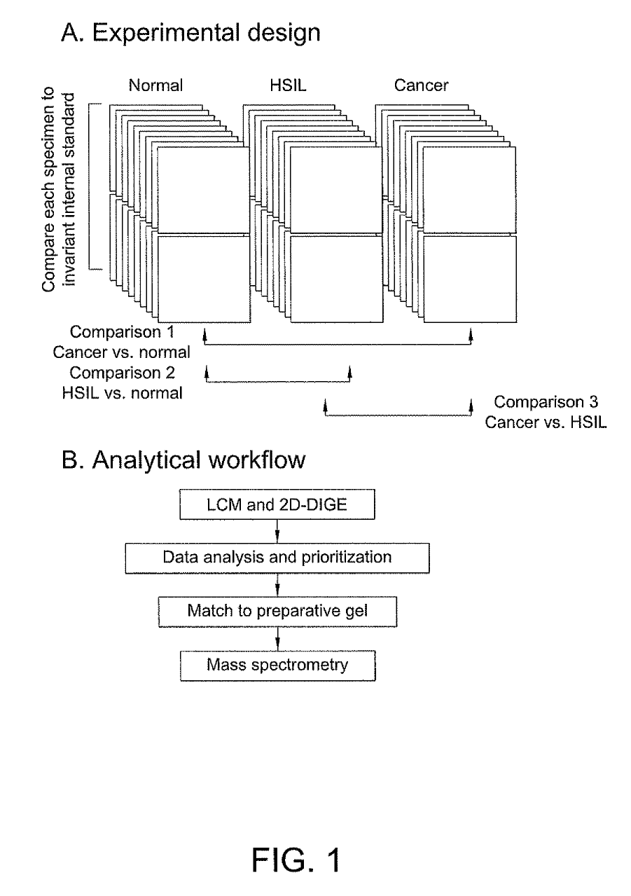

There were three experimental groups: normal, patient-matched HSIL, and cancer (FIG. 1). Specimens were obtained from the Instituto Nacional de Enfermedades Neoplásicas (INEN,Lima, Peru). Patients who had positive Pap smears and were scheduled to undergo gynecologic surgery were eligible. Following institutional review board guidelines, subjects were asked to provide informed consent for use of their tissue in research. Patients with a finding of HSIL contributed both lesional tissue and normal tissue from elsewhere in the cervix. Patients with a finding of invasive cancer contributed lesional tissue only (typically, no normal anatomy remained). Three comparisons were made: (1) cancer vs. normal (unpaired), (2) HSIL vs. normal (paired), and (3) cancer vs. HSIL (unpaired). Tissues were snap frozen, and epithelial or lesional tissue was later collected by LCM as described [23]. An invariant internal standard was prepared as a mixture of normal cervical tissue from a patient who underwent transabdominal hysterectomy for symptomatic leiomyomas and cervical squamous cell carcinoma from a different patient who underwent radical hysterectomy. Samples and an internal standard were labeled with different dyes, so the abundance of each spot could be quantified relative to the corresponding spot in the internal standard [27]. Candidate biomarkers were ranked using the Significance Analysis of Microarrays (SAM, version 3.0) add-in for Microsoft Excel (available at http://www-stat.stanford.edu/˜tibs/SAM/). An FDR of 10% was used as a threshold cutoff for each spot.

LCM and 2D-DIGE

Frozen sections (5 μm) were stained briefly with Nuclear Fast Red and LCM was performed using an Arcturus PixCell IIe microscope. Caps, with polymer film and adherent cells, were placed onto a microcentrifuge tube containing lysis buffer (7 M urea, 2 M thiourea, 4% CHAPS, 0.4 mM AEBSF (protease inhibitor), 40 mM Tris-HCl pH 8, 5 mM Mg(OAc)2). Tubes were inverted to wet the polymer film and incubated for 30 min at room temperature. The resulting extracts were sonicated five times for 30 sec each and centrifuged at 14,000 g for 15 min, and the supernatant was transferred to a fresh tube. Quantities of labeling reagents were based on the estimate that 5000 cell samples contained about 1 μg of extractable protein. Tris-(2-carboxyethyl)-phosphine (TCEP) was added (0.4 nmol), and the mixture was incubated 1 h at 37° C. Cy5 sulfhydryl-reactive dye was added (0.8 nmol, GE Healthcare, Buckinghamshire, UK) and incubation was continued for 30 min at 37° C. The reaction was terminated by addition of an equal volume of 2× sample buffer (7 M urea, 2 M thiourea, 4% CHAPS, 130 mM dithiothreitol, 2% ampholytes). After 15 min at 4° C., the sample was diluted to a final volume of 450 μl with rehydration buffer (7 M urea, 2 M thiourea, 4% CHAPS, 13 mM dithiothreitol, 1% ampholytes). The samples were stored frozen at −80° C. until use. Although freezing and thawing of samples reduced the quality of 2D gels in our previous study, saturation labeling prior to freezing protects against this effect, perhaps by blocking oxidation of free cysteines. The internal standard was prepared by grinding bulk tissue under liquid nitrogen. Proteins were solubilized as described for LCM samples. Protein concentration was measured, and the samples were saturation-labeled with Cy3 using the same ratio of dye and TCEP to protein as for the LCM samples.

A mixture of Cy5-labeled sample and Cy3-labeled internal standard was loaded into a 24 cm strip holder containing a pH 3-10 nonlinear IPG strip and overlaid with Immobiline DryStrip Cover Fluid (GE Healthcare), Rehydration was carried out for 15 h at 20° C. with an applied electric field of 30 V. For first-dimension electrophoresis, electric potentials of 500 V for 1 h, 1000 V for 2 h, and 8000 V for 7 h were applied. The strip was removed and equilibrated twice in 6 M urea, 100 mM Tris-HCl pH 8, 2% SDS, 32.5 mM dithiothreitol, and 30% glycerol for 15 min at room temperature. The strip was applied to the top of a 12.5% SDS gel (25 cm×20 cm×0.1 cm), and electrophoresis was performed using 10 mA per gel overnight. The gel was removed and scanned using a GE Healthcare Typhoon 9400 Series Variable Imager.

Preparative Gel for Protein Identification

For the preparative gel, a volume of cervical tissue protein lysate containing 500 μg of internal standard in lysis buffer was labeled in a reaction containing 20 mM Cy3 sulfhydryl-reactive dye, 1× sample buffer (7 M urea, 2 M thiourea, 4% CHAPS), 1% Pharmalytes, and 130 mM dithiothreitol in a final volume of 450 μl. The mixture was loaded into a 24 cm strip holder containing a pH 3-10 nonlinear IPG strip and overlaid with Immobiline DryStrip Cover Fluid. The sample was separated in the first and second dimensions as described above. The gel was scanned, and then fixed with 30% methanol and 7.5% acetic acid solution. The preparative gel image was matched to the analytical gel images to obtain coordinates for spots of interest. Protein plugs were robotically cored and transferred to a 96 well plate for tryptic digestion.

Preparative Gel and Mass Spectroscopy

Spots of interest were matched to a preparative gel, and proteins were identified by mass spectrometry [23]. Following trypsin digestion, extracted peptides were spotted onto a 192-well MALDI-TOF target plate for the Applied Biosystems Incorporated (ABI) 4700 Proteomics Analyzer. Automated MALDI-TOF mass spectrometry provided a peptide mass fingerprint. In addition, for each analysis the 20 most prominent peptides (excluding trypsin peaks) were subjected to collision-induced dissociation to obtain sequence information. Spectra were searched using the GPS Explorer (ABI) search tool and Mascot algorithm (Matrix Biosciences) against the NCBInr protein database.

Immunohistochemistry

Immunohistochemistry was performed using 6 μM replicate frozen sections from normal, patient-matched HSIL, and carcinoma samples (n=3 per group). Slides were air dried, fixed in 10% neutral buffered formalin for 5 min, and rinsed with distilled water. Endogenous peroxidase was quenched by incubating twice in 0.3% H₂O₂ for 5 min., then washing twice in PBS for 5 min. Slides were blocked with normal donkey serum for 20 min, then incubated with the following primary antibodies for 30 min: 1:100 anti-cornulin (Alexis, San Diego, Calif.), 1:1000 anti-Hsp27 (HSPB1) (Assay Designs, Ann Arbor, Mich.), 1:1000 anti-Manganese Superoxide Dismutase 2 (Abeam, Cambridge, UK), or 1:100 anti-PA28β (Abnova, Taipei, Taiwan). Slides were washed twice with PBS, then with HRP-conjugated goat anti-rabbit immunoglobulin (cornulin and superoxide dismutase), or goat anti-mouse immunoglobulin (PA28β and Hsp27) (Envision+HRP kit, Dako Corp. Carpinteria, Calif.). Slides were rinsed twice with PBS, and bound antibody was detected using diaminobenzidine. Slides were counterstained with hematoxylin. Scoring was determined by a board-certified pathologist.

Commercial tissue microarrays containing histologically confirmed cervical tissue from a variety of disease states were purchased from Biomax, Inc. (Rockville, Md.). Each microarray contained 30 carcinoma specimens, 10 CIN specimens, 10 inflamed cervical tissue specimens, and 10 normal specimens. Slides were deparaffinized and run through graded alcohols to distilled water. Slides were pretreated with Target Retrieval Solution PH 6.0, (Dako Corp, Carpinteria, Calif.) using a steamer (Black and Decker rice steamer) and rinsed in distilled water. Antibody staining and development were the same as for the frozen sections.

### Results

Pilot sections for each specimen were stained with hematoxylin and eosin to reveal morphological detail. HSIL samples demonstrated >90% involvement of the epithelium with high-grade dysplastic cells that had not invaded through the basement membrane. Cervical cancer samples demonstrated moderately differentiated, nonkeratinizing squamous cell carcinomas. All preparative sections were stained with Nuclear Fast Red for LCM. The more intensely stained epithelial or lesional tissue was collected, leaving behind the lighter-stained stroma.

Evaluation of the technical reproducibility of the combined LCM and 2D-DIGE procedure and power analysis was performed. Independent LCM sampling of normal cervical tissue and cervical cancer was carried out. Protein abundance was analyzed by 2DDIGE using an invariant internal standard, and evaluated reproducibility based on coefficients of variation. The median coefficient of variation was 23% for both normal cervical tissue and cervical cancer. Because the analytical methodology is the same, the distribution of coefficients for the HSIL group should be the same, although it was not possible to perform the same replicate sampling because of the small size and scarcity of the lesions.

To estimate statistical power for biomarker discovery, a hypothetical marker with a between-group difference of 2-fold and a CV of 30% for technical variation was considered, both of which were within the observed range. Within-group biological variation would be on the same order as technical variation and that tests would be conducted on the logscale so that the CV roughly corresponds to the standard deviation of the log-transformed data. A study would require 10 subjects per group to obtain 80% power to identify such features using a two-sided alpha of 0.05.

For the main analysis, proteins from the 30 samples (n=10 per group) were extracted, labeled, and analyzed by 2D-DIGE. An average of 2257 spots was identified in each gel, of which an average of 1489 spots was matched to the master map. Of these, 135 spots were selected for further analysis, based on manual inspection showing unequivocal alignment across spot maps generated from all 30 samples. To prioritize spots for analysis, protein abundance values were calculated as described in Materials and Methods and used as input data for Significance Analysis of Microarray (SAM). For each of the three comparisons (cancer versus normal, HSIL versus normal, and cancer versus HSIL), SAM calculated a relative difference score, d(i), and a false discovery rate based on analysis of permuted data sets. A threshold value for d score based on a false discovery rate (FOR) of 10% or less and an additional filter to exclude spots with an absolute change in expression level of <2.0-fold was applied. Tissue biomarkers with changes of <2.0-fold might be difficult to measure reliably in a clinical laboratory (e.g., by immunohistochemistry) and thus would be unlikely to be widely adopted. Application of a filter based on fold change has been shown to further reduce FDR [28]. Based on these criteria, 53 features (spots) were identified as candidate biomarkers.

### Example 2

### Proteomic Patterns in Normal, HSIL, and Cancer

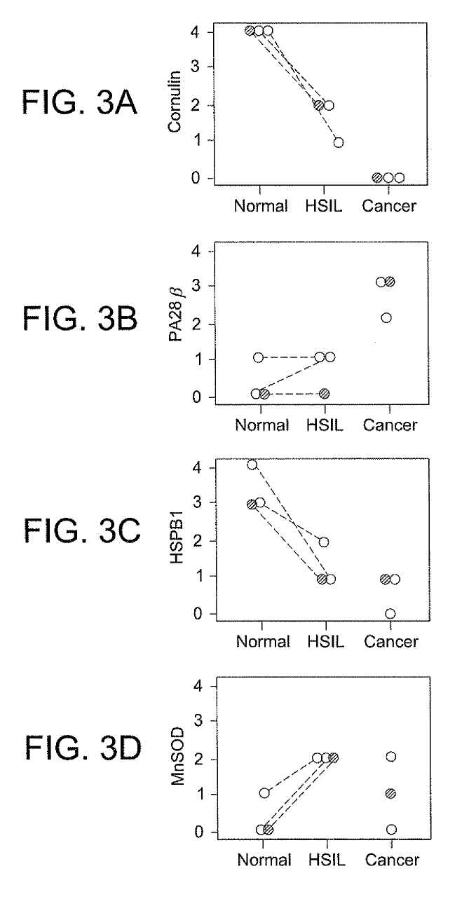

Based on the SAM analysis, there were 42 spots that distinguished cancer from normal, 23 that distinguished HSIL from normal, and 9 that distinguished cancer from HSIL. Some spots were significant in two or more of these pairwise comparisons (20/53) and one distinguished all three sample groups. Individual data values for four representative markers are presented in FIG. 3A-D. The vertical axis represents the “internal ratio” (IR) of expression for each spot relative to the internal standard in the same gel. Data are plotted as log2 IR, such that each unit on the vertical axis corresponds to a 2-fold change. Dashed lines, which connect paired normal and HSIL specimens from the same patient, illustrate how the availability of paired samples reveal consistent expression trends that might not otherwise have been apparent. Viewing group means, in addition to the individual values, provides additional insight. HSIL has its own, distinctive, pattern of expression, with some markers more cancer-like, and others more normal-like.

### Example 3

### Match to Preparative Gel and Mass Spectrometry Analysis

To identify spots at the molecular level, a separate preparative gel was run with 500 μg of Cy3-labeled mixed internal standard, matched the spot map to the master map from the analytical gels, picked spots of interest, and obtained mass spectrometry identifications as described in Example 1. 31 spots were picked including only those that could be unambiguously matched between the preparative gel and the master map and that were well resolved from abundant neighboring spots, and obtained definite identifications for 29. Among these, there were five instances where nearby, co-regulated spots proved to be the same protein, leaving the 23 unique proteins listed in Table 1. Many of the proteins are known by more than one name; when possible systematic nomenclature that reflects identities of proteins as members of gene families was used, with synonyms listed only when they are widely used in the literature. Mascot scores from peptide mass fingerprinting and collisionally-induced dissociation were greater than 80 (60 is the threshold for significance), and all protein identifications achieved a 100% protein score confidence interval. MS coverage of 30% and sequence information from 15 peptides unequivocally identified this protein as the differentiation marker, cornulin. Calculated mass and pI values were consistent with migration. The identified proteins have a diverse set of mass and pI values, indicating that the selection criteria for potential biomarkers did not introduce any obvious bias with respect to protein size or charge.

### Example 4

### Literature Review

As a first step in understanding the significance of the findings, a literature review was performed to identify relevant genetic, structural, and biological data for each protein. Several of the cytokeratins, two of the detoxifying enzymes, HSPB1, and Serpin 3 (SCCA1), have all been previously characterized in the context of cervical cancer development). About half of the candidate markers, however, had not been previously associated with cervical cancer or HSIL. In many cases, biomarker increase and decrease can be rationalized in terms of the known effects of HPV E6 and E7 oncoproteins, selection for growth advantage during latency, or host lesion interactions.

### Example 5

### Validation by Immunohistochemical Staining

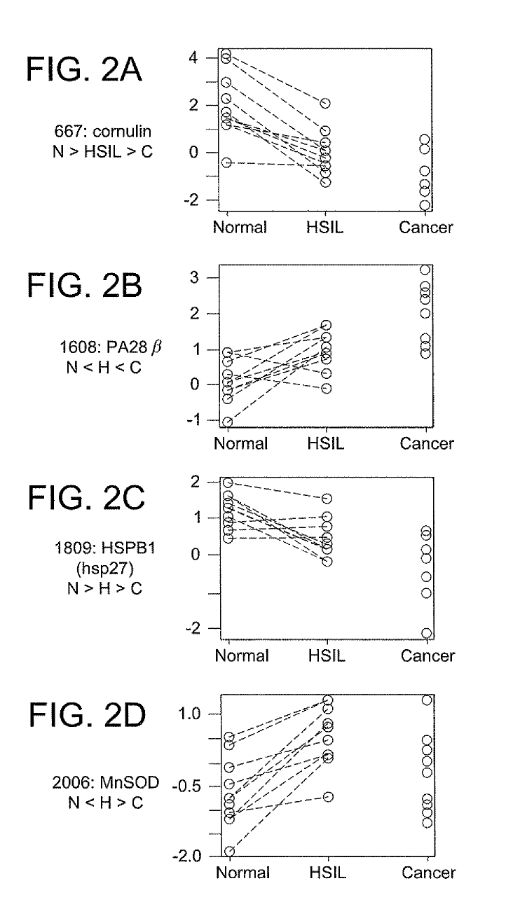

To increase confidence in the 2-DICE and mass spectrometry findings, three specimens from each group in the original cohort were randomly selected and immunohistochemistry was performed on them. Four markers were investigated because of the commercial availability of antibodies suitable for immunochemistry and because the markers were (a) novel in the context of cervical cancer or (b) there was a discrepancy between the data and previous reports. Serial sections were stained with antibodies to cornulin, PA28β, HSPB1 and MnSOD and the slides were scored on a standard scale of 0 to 3 based on intensity of staining (FIGS. 3A-D). Results showed generally good agreement with the 2D-DIGE quantification (compare FIGS. 2A-D with FIGS. 3A-D). Prominent cornulin staining is evident in the maturing squamous cells of the normal epithelium, in only a thin rim of non-dysplastic cells representing the outermost layer of epithelium in HSIL, and not at all in an invasive cancer sample. PA28β staining was evident only in cancer, and not in normal or HSIL. The pattern of HSPB1 staining was similar to cornulin, with intense staining in the normal epithelium, consistent with reports that this small heat shock protein is a cornification chaperone [29, 30]. HSPB1 staining was also present, but at a lower level in HSIL and cancer (panel C), consistent with a prior immunohistochemical study of HSPB1 expression in cervical pre-cancerous lesions and cancer [31]. There was HSPB1 staining in areas of necrosis in cancer samples (not shown) but necrotic areas were excluded in the LCM procedure and thus not represented in the 2D-DIGE sampling. Immunohistochemical staining of MnSOD showed expression in a thin layer of cells along the basal layer of the normal squamous epithelium, an increase in expression in HSIL, and somewhat of a decline in cancer, again consistent with 2D-DIGE.

To increase statistical power and extend the findings to a different cohort of patients, additional immunohistochemistry experiments were performed using formalin fixed paraffin embedded tissue microarrrays (FIGS. 4A-B). The microarrays include more patients (n=60) and additional experimental groups (e.g., benign inflammation and lower grades of cervical intraepithelial neoplasia (CIN)). Tissue microarrays were stained with anti-cornulin or anti-Hsp27 (HSPB1), Staining intensity was scored on the same 0 to 3 scale. Statistical analysis was performed by one-way ANOVA. Differences contributing to group variance were calculated in pair-wise comparisons using the Tukey's Honestly Significant Difference Test.

Anti-cornulin staining (FIG. 4A) showed no apparent difference between normal and inflamed tissue, but a highly significant difference between these two groups and cancer (p<0.001). The CIN samples had a wide distribution of values centered in between normal and cancer. The variance was attributed to the presence of multiple grades of CIN in this cohort. Because of the within-group variance, comparisons of CIN to the other groups did not reveal a statistically significant difference.

Anti-HSPB1 staining confirmed that expression of this molecular chaperone is high in normal epithelium, inflamed tissue, and HSIL, consistent with results obtained with 2D-DIGE. Surprisingly, expression in cancer was far more variable than in the original cohort. This was particularly true of grade 3 cancers, where HSPB1 was present either in high amounts or not at all.

### Example 6

### Reproducibility of Combined LCM and 2D-DIGE Analysis

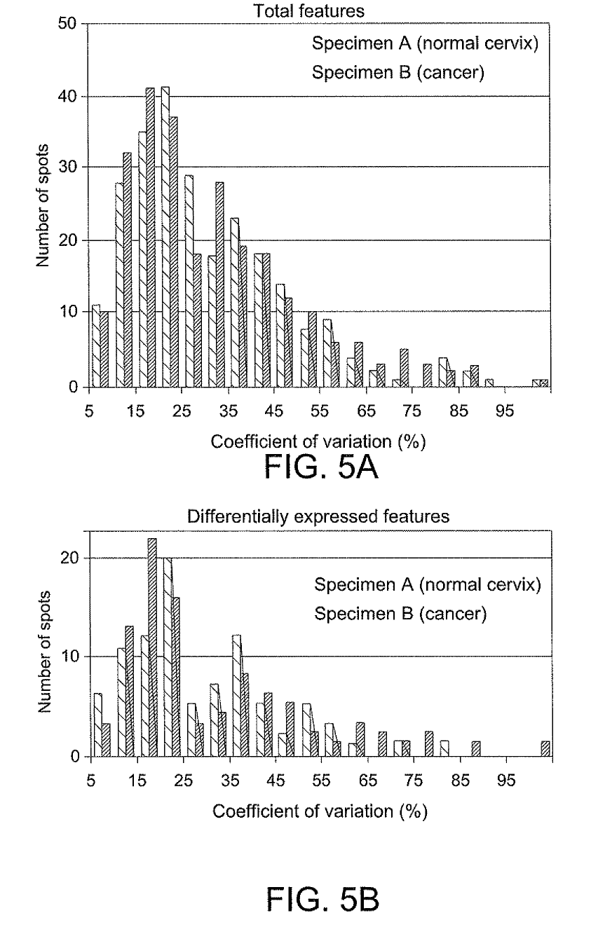

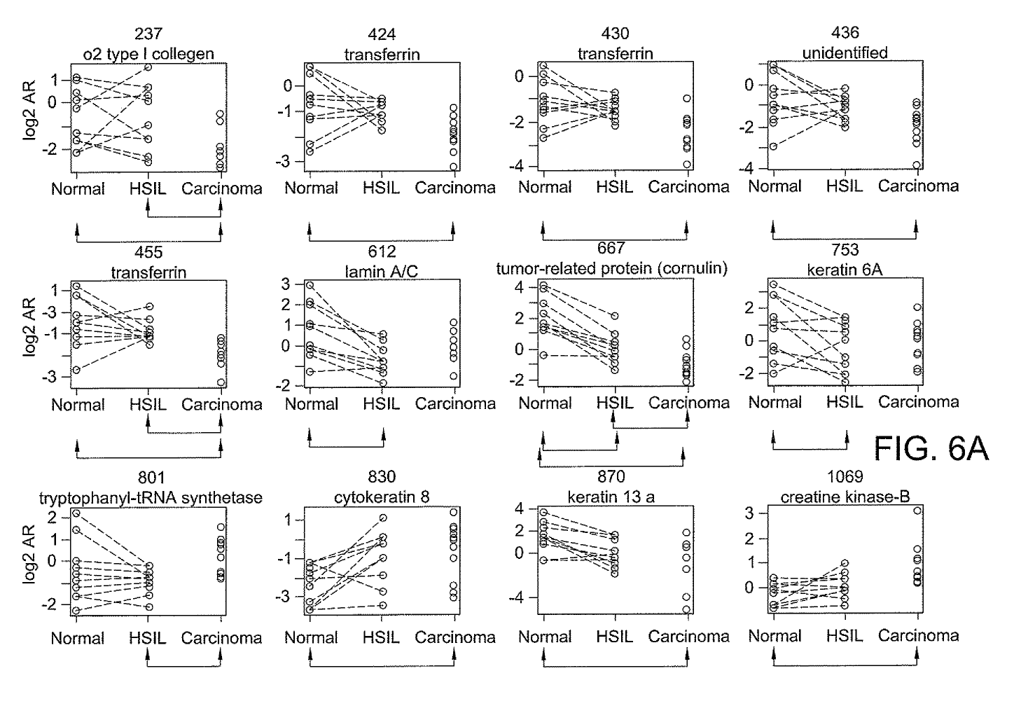

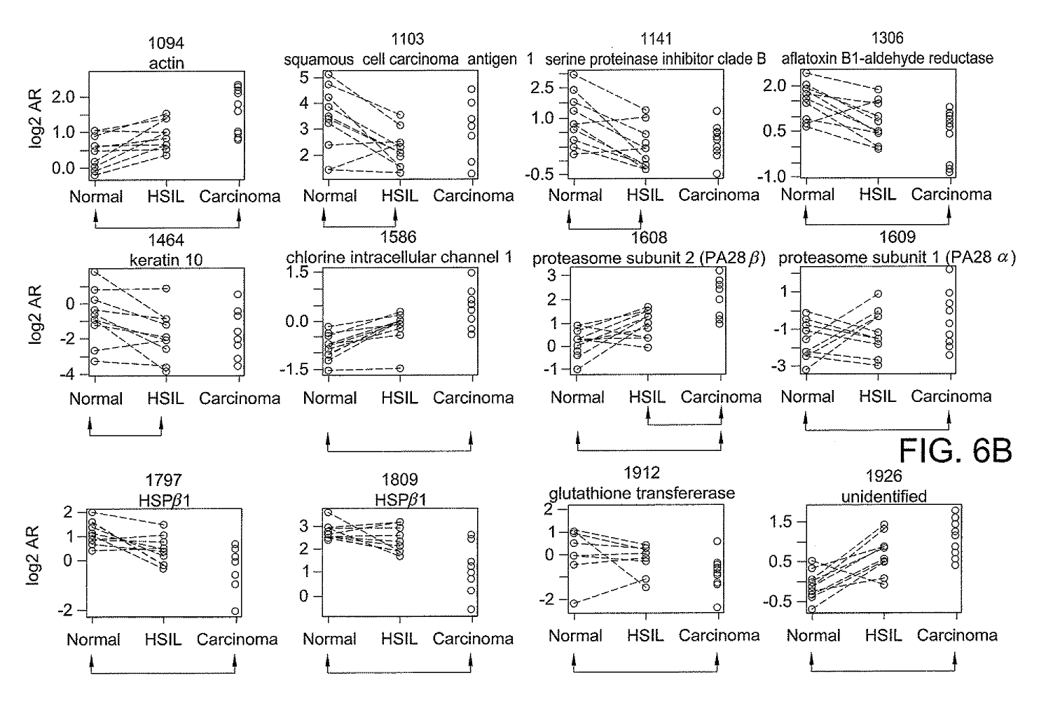

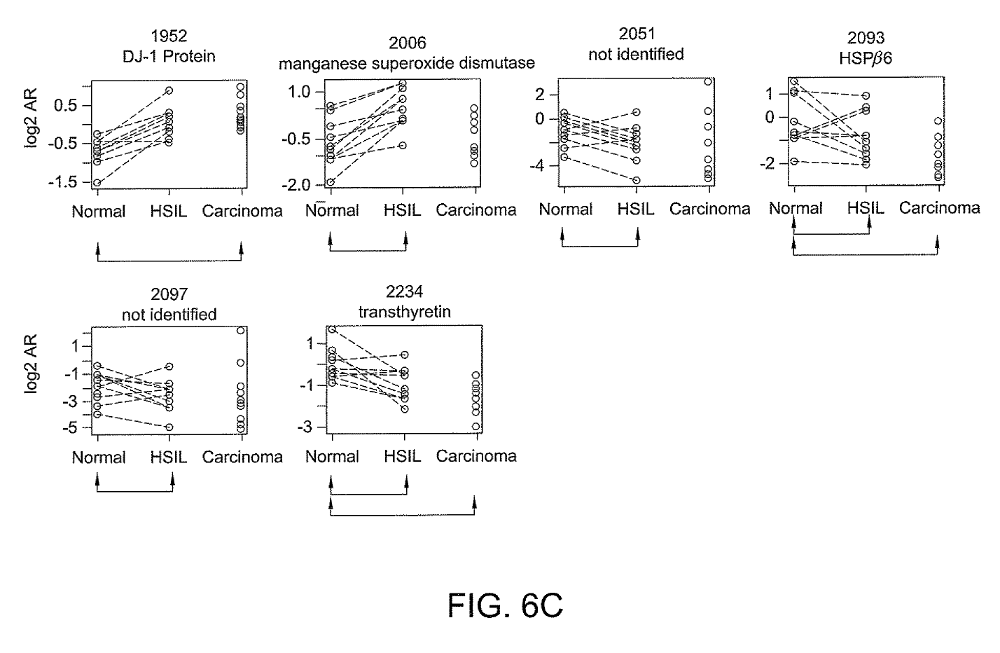

A pilot experiment was performed to analyze the reproducibility of combined LCM and 2DDIGE analysis. Specimens A and B consisted of a normal epithelium and a malignancy from human cervix. Each specimen was sampled in triplicate by LCM and analyzed proteins by 2DDIGE. A pooled internal standard prepared by macroscopic dissection of tissue blocks representing the two specimens was used. Protein spots based on their presence in all six internal standard images were matched. 261 spots across the entire data set were matched. For each spot in each gel, the abundance relative to the same feature in the internal standard was determined to derive an “internal ratio” (IR). For each feature, the mean IR was determined as well as a within-group coefficient of variation (CV). A histogram showing the distribution of CVs is shown in FIG. 5. The median CVs for Specimens A (normal cervical epithelium) and B (squamous cervical carcinoma) were 23.1% and 22.7%, respectively.

Only a fraction of the features in a proteomic data set are potential biomarkers since the majority of the features are invariant between sample groups. To focus on the most relevant subset of the data, 94 features with 2-fold or greater differences between normal and cancer were considered as potential biomarkers. The distribution of CVs in this subset was similar to that in the full data set (Panel B). Although the 2-fold cutoff is arbitrary (chosen for exploratory purposes and not based on explicit statistical reasoning) the use of a different threshold would not materially alter the overall conclusion that the distribution for CVs among potential biomarkers is similar to that in the data set as a whole.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

TABLE 1
Identified Proteins Ranked by d Score

| Spot Number | Accession Number | Protein Name | Mass (kD a) | Pl | Peptide (coverage0 b | Mascot score c) | Comparison (CN) d) |
|---|---|---|---|---|---|---|---|
| 667 | Q9UBG3 | Cornulin | 53 | 5.73 | 15 (33%) | 143 | (7.8)* |
| 1608 | Q9UL46 | PA28 β | 27 | 5.44 | 8 (40%) | 382 | 3.6** |
| 1926 | Q99497 | DJ-1 protein | 20 | 6.33 | 5 (30%) | 83 | 2.1** |
| 1094 | P63261 | Actin | 40 | 5.55 | 12 (37%) | 317 | 2.5** |
| 2234 | P02766 | Transthyretin | 13 | 5.57 | 9 (80%) | 361 | (3.0)* |
| 1809 | P04792 | HSPB1 | 22 | 7.83 | 9 (46%) | 289 | (2.8)* |
| 1586 | Q5SRT3 | Cl⁻ intracellular | 26 | 4.95 | 7 (29%) | 179 | 2.3** |
| 830 | P05787 | Cytokeratin 8 | 53 | 5.52 | 14 (32%) | 141 | 3.6** |
| 455 | P02787 | Transferrin | 55 | 6.00 | 7 (21%) | 106 | (2.8)* |
| 2093 | Q14558 | Hspβ6 (HSP20) | 16 | 5.95 | 5 (38%) | 162 | (2.8)* |
| 1306 | Q43488 | Aflatoxin | 40 | 6.70 | 8 (30%) | 227 | (2.2)* |
| | | reductase | | | | | |
| 237 | P08123 | α2 type 1 | 12 | 9.08 | 11 (10%) | 175 | (2.6)* |
| | | Collagen | | | | | |
| 1069 | P12277 | Creatine kinase β | 42 | 5.34 | 15 (30%) | 609 | 2.1** |
| 870 | P13646 | Cytokeratin 13 | 49 | 4.87 | 19 (34%) | 514 | (4.1)* |
| 1912 | P09211 | GST π | 23 | 5.43 | 10 (62%) | 125 | (2.1)* |
| 1609 | Q06323 | PA28 α | 28 | 5.78 | 14 (54%) | 99 | 2.3** |
| 2006 | P04179 | Manganese | 22 | 6.86 | 7 (37%) | 89 | 1.6 |
| | | SQD | | | | | |
| 612 | Q57CJ3 | Lamin A/C | 65 | 6.40 | 19 (30%) | 80 | (1.6) |
| 1141 | P30740 | Serpin B1 | 42 | 5.90 | 18 (44%) | 402 | (1.2) |
| | | (elastase) | | | | | |
| 1103 | Q81X13 | Serpin B3 | 44 | 6.35 | 16 (52%) | 546 | (1.7) |
| | | (SCCA1) | | | | | |
| 1464 | P13645 | Cytokeratin60 | 59 | 5.09 | 14 (22%) | 122 | (2.1)* |
| | | 1D | | | | | |
| 753 | P02538 | Cytokeratin 6A | 60 | 7.59 | 18 (31%) | 274 | (1.8) |
| 801 | P23381 | Trp-tRNA | 53 | 6.03 | 6 (35%) | 235 | 1.7 |
| | | synthetase | | | | | |

| | | | | | | | Abs (d | FDR |
|---|---|---|---|---|---|---|---|---|
| Spot Number | Abs (d score) e) | FDR (%) f) | Comparison 2 (HN) | Abs (d score) | FDR (%) | Comparison 3 (CH) | score) | (%) |
| 667 | 4.0* | * | (3.9)* | 4.1* | * | (2.0) | 1.6* | 4.7* |
| 1608 | 3.3** | ** | 1.8 | 2.0 | 3.0 | 2.0* | 1.9* | 7.5* |
| 1926 | 2.7** | ** | 1.7 | 2.7 | — | 1.3 | 2.0 | 31 |
| 1094 | 2.8** | ** | 1.4 | 1.8 | 3.0 | 1.8 | 1.9 | 7.5 |
| 2234 | 2.8* | * | (2.0)* | 2.1* | * | (1.5)* | 1.1 | 7.5 |
| 1809 | 2.7* | * | (1.3) | 1.2 | 12 | (2.2)* | 2.0* | 0* |
| 1586 | 2.6** | ** | 1.6 | 2.4 | — | 1.4 | 1.1 | 20 |
| 830 | 2.4** | ** | 3.2** | 2.52** | ** | 1.1 | 0.22 | 49 |
| 455 | 2.3* | * | (1.3) | 0.77 | 16 | (2.1)* | 2.2* | * |
| 2093 | 2.3* | * | (1.6) | 1.1 | 9.0 | (1.8) | 1.4 | 5.9 |
| 1306 | 2.1* | * | (1.6) | 2.02 | — | (1.3) | .76 | 19 |
| 237 | 2.0* | * | (1.0) | 0.080 | 39 | (2.6)* | 2.0* | * |
| 1069 | 2.0** | 1.0** | 1.2 | 0.71 | 32 | 1.8 | 1.4 | 16 |
| 870 | 1.9* | * | (2.6)* | 2.6* | * | (1.6) | 0.61 | 23 |
| 1912 | 1.6* | * | (1.2) | 0.56 | 20 | (1.7) | 1.4 | 5.9 |
| 1609 | 1.6** | 2.8** | (1.3) | 0.62 | 32 | 1.8 | 1.0 | 23 |
| 2006 | 0.40 | 16 | 2.3** | 3.3** | ** | (1.8) | 1.5 | 4.7 |
| 612 | 1.0 | 5.3 | (2.6)* | 2.6* | * | 1.6 | 1.2 | 19 |
| 1141 | 1.3 | 1.9 | (2.1)* | 2.4* | * | 1.9 | 0.47 | 45 |
| 1103 | 0.29 | 16 | (2.0)* | 2.2* | * | 1.2 | 1.5 | 16 |
| 1464 | 1.3* | 1.9* | (2.1)* | 2.1* | * | (1.0) | 0.044 | 49 |
| 753 | 0.88 | 6.7 | (2.5)* | 1.8* | 1.6* | 1.4 | 0.56 | 43 |
| 801 | 0.97 | 13 | (1.4) | 0.98 | 13 | 2.4** | 2.3** | 7.1** |

a) Spots were ranked in order of decreasing absolute value of d score as determined by SAM algorithm. Dark grey shading denotes up-regulation in the indicated comparison, light grey shading denotes down-regulation, lack of shading denotes not significant. Spot numbers are as they appear on the master analytical gel. Protein accession numbers are from the SwissProt database. Predicted protein masses and isoelectric points are based on conceptual translation. CN: comparison of cancer and normal. HN: comparison of HSIL and normal. CH: comparison of cancer and HSIL.
b) Number of peptides that matched the identified protein sequence, followed by percent sequence coverage.
c) Mascot score based on combined peptide mass fingerprinting and masses of collisionally-induced dissociation peptides
d) Fold change in expression. Values in parentheses are decreases, other values are increases.
e) Absolute value of d(i) from SAM calculation.
f) False discovery rate from SAM calculation.

### REFERENCES

[1] Sankaranarayanan, R., Ferlay, J., Worldwide burden of gynaecological cancer: the size of the problem. Best Pract Res Clin Obstet Gynaecol 2006, 20, 207-225.
[2] Bray, F., Loos, A. H., McCarron, P., Weiderpass, E., et al., Trends in cervical squamous cell carcinoma incidence in 13 European countries: changing risk and the effects of screening. Cancer Epidemiol Biomarkers Preb 2005, 14, 677-686.
[3] Landis, S. H., Murray, T., Bolden, S., Wingo, P. A., Cancer statistics, 1999. CA Cancer J Clin 1999, 49, 8-31.
[4] Solomon, D., Davey, D., Kurman, R., Moriarty, A., et al., The 2001 Bethesda System: terminology for reporting results of cervical cytology. Jama 2002, 287, 2114-2119.
[5] Nanda, K., McCrory, D. C., Myers, E. R., Bastian, L. A., et al., Accuracy of the Papanicolaou test in screening for and follow-up of cervical cytologic abnormalities: a systematic review. Ann Intern Med 2000, 132, 810-819.
[6] Malpica, A., Matisic, J. P., Niekirk, D. V., Crum, C. P., et al., Kappa statistics to measure interrater and intrarater agreement for 1790 cervical biopsy specimens among twelve pathologists: qualitative histopathologic analysis and methodologic issues. Gynecol Oncol 2005, 99, S38-52.
[7] Cantor, S. B., Atkinson, E. N., Cardenas-Turanzas, M., Benedet, J. L., et al., Natural history of cervical intraepithelial neoplasia: a meta-analysis. Acta Cytol 2005, 49, 405-415.
[8] Sadler, L., Saftlas, A., Wang, W., Exeter, M., et al., Treatment for cervical intraepithelial neoplasia and risk of preterm delivery. JAMA 2004, 291, 2100-2106.
[9] Samson, S. L., Bentley, J. R., Fahey, T. J., McKay, D. J., Gill, G. H., The effect of loop electrosurgical excision procedure on future pregnancy outcome. Obstet Gynecol 2005, 105, 325-332.
[10] Doorbar, J., Molecular biology of human papillomavirus infection and cervical cancer. Clin Sci (Lond) 2006, 110, 525-541.
[11] Munger, K., Basile, J. R., Duensing, S., Eichten, A., et al., Biological activities and molecular targets of the human papillomavirus E7 oncoprotein. Oncogene 2001, 20,7888-7898.
[12] Arbyn, M., Buntinx, F., Van Ranst, M., Paraskevaidis, E., et al., Virologic versus cytologic triage of women with equivocal Pap smears: a meta-analysis of the accuracy to detect high-grade intraepithelial neoplasia. J Natl Cancer Inst 2004, 96, 280-293.
[13] Kong, C. S., Balzer, B. L., Troxell, M. L., Patterson, B. K., Longacre, T. A., p16INK4A immunohistochemistry is superior to HPV in situ hybridization for the detection of highrisk HPV in atypical squamous metaplasia. Am J Surg Pathol 2007, 31, 33-43.
[14] El-Ghobashy, A. A., Shaaban, A. M., Innes, J., Prime, W., Herrington, C. S., Differential expression of cyclin-dependent kinase inhibitors and apoptosis-related proteins in endocervical lesions. Eur J Cancer 2007, 43, 2011-2018.
[15] Baak, J. P., Kruse, A. J., Robboy, S. J., Janssen, E. A., et al., Dynamic behavioural interpretation of cervical intraepithelial neoplasia with molecular biomarkers. J Clin Pathol 2006, 59, 1017-1028.
[16] Dunne, E. F., Unger, E. R., Sternberg, M., McQuillan, G., et al., Prevalence of HPV infection among females in the United States. Jama 2007, 297, 813-819.
[17] Bae, S. M., Lee, C. H., Cho, Y. L., Nam, K. H., et al., Two-dimensional gel analysis of protein expression profile in squamous cervical cancer patients. Gynecol Oncol 2005, 99, 26-35.
[18] Choi, Y. P., Kang, S., Hong, S., Xie, X., Cho, N. H., Proteomic analysis of progressive factors in uterine cervical cancer. Proteomics 2005, 5, 1481-1493.
[19] Gu, Y., Wu, S. L., Meyer, J. L., Hancock, W. S., et al., Proteomic Analysis of High-Grade Dysplastic Cervical Cells Obtained from ThinPrep Slides Using Laser Capture Microdissection and Mass Spectrometry. Journal of Proteome Research 2007.
[20] Melnikow, J., Nuovo, J., Willan, A. R., Chan, B. K., Howell, L. P., Natural history of cervical squamous intraepithelial lesions: a meta-analysis. Obstet Gynecol 1998, 92, 727-735.
[21] Shaw, J., Rowlinson, R., Nickson, J., Stone, T., et al., Evaluation of saturation labeling two-dimensional difference gel electrophoresis fluorescent dyes. Proteomics 2003, 3, 1181-1195.
[22] Sitek, B., Potthoff, S., Schulenborg, T., Stegbauer, J., et al., Novel approaches to analyse glomerular proteins from smallest scale murine and human samples using DIGE saturation labelling. Proteomics 2006, 6, 4337-4345.
[23] Greengauz-Roberts, O., Stoppler, H., Nomura, S., Yamaguchi, H., et al., Saturation labeling with cysteine-reactive cyanine fluorescent dyes provides increased sensitivity for protein expression profiling of laser-microdissected clinical specimens. Proteomics 2005, 5, 1746-1757.
[24] Wilson, K. E., Marouga, R., Prime, J. E., Pashby, D. P., et al., Comparative proteomic analysis using samples obtained with laser microdissection and saturation dye labelling. Proteomics 2005, 5, 3851-3858.
[25] Shekouh, A. R., Thompson, C. C., Prime, W., Campbell, F., et al., Application of laser capture microdissection combined with two-dimensional electrophoresis for the discovery of differentially regulated proteins in pancreatic ductal adenocarcinoma. Proteomics 2003, 3, 1988-2001.
[26] Seike, M., Kondo, T., Fujii, K., Yamada, T., et al., Proteomie signature of human cancer cells. Proteomics 2004, 4, 2776-2788.
[27] Alban, A., David, S. O., Bjorkesten, L., Andersson, C., et al., A novel experimental design for comparative two-dimensional gel analysis: two-dimensional difference gel electrophoresis incorporating a pooled internal standard. Proteomics 2003, 3, 36-44.
[28] Tusher, V. G., Tibshirani, R., Chu, G., Significance analysis of microarrays applied to the ionizing radiation response. Proc Natl Acad Sci USA 2001, 98, 5116-5121.
[29] Jonak, C., Klosner, G., Kokesch, C., D, F. O., et al., Subcorneal colocalization of the small heat shock protein, hsp27, with keratins and proteins of the cornified cell envelope. Br J Dermatol 2002, 147, 13-19.
[30] O'Shaughnessy, R. F., Welti, J. C., Cooke, J. C., Avilion, A. A., et al., AKT-dependent HspB1 (Hsp27) activity in epidermal differentiation. J Biol Chem 2007, 282, 17297-17305.
[31] Ciocca, D. R., Lo Castro, G., Alonio, L. V., Cobo, M. F., et al., Effect of human papillomavirus infection on estrogen receptor and heat shock protein hsp27 phenotype in human cervix and vagina. Int J Gynecol Pathol 1992, 11, 113-121.
[32] Lee, K. A., Kang, J. W., Shim, J. H., Kho, C. W., et al., Protein profiling and identification of modulators regulated by human papillomavirus 16 E7 oncogene in HaCaT keratinocytes by proteomics. Gynecol Oncol 2005, 99, 142-152.
[33] Lee, K. A., Shim, J. H., Kho, C. W., Park, S. G., et al., Protein profiling and identification of modulators regulated by the E7 oncogene in the C33A cell line by proteomics and genomics. Proteomics 2004, 4, 839-848.
[34] Rahman-Roblick, R., Roblick, U. J., Hellman, U., Conrotto, P., et al., p53 targets identified by protein expression profiling. Proc Natl Acad Sci USA 2007, 104, 5401-5406.
[35] Zhao, J., Schmieg, F. I., Simmons, D. T., Molloy, G. R., Mouse p53 represses the rat brain creatine kinase gene but activates the rat muscle creatine kinase gene. Mol Cell Biol 1994, 14, 8483-8492.
[36] Contzler, R., Favre, B., Huber, M., Hohl, D., Cornulin, a new member of the “fused gene” family, is expressed during epidermal differentiation. J Invest Dermatol 2005, 124, 990-997.
[37] Darragh, J., Hunter, M., Pohler, E., Nelson, L., et al., The calcium-binding domain of the stress protein SEP53 is required for survival in response to deoxycholic acid-mediated injury. Febs J 2006, 273, 1930-1947.
[38] Manavi, M., Hudelist, G., Fink-Retter, A., Gschwandtler-Kaulich, D., et al., Gene profiling in Pap-cell smears of high-risk human papillomavirus-positive squamous cervical carcinoma. Gynecol Oncol 2007, 105, 418-426.
[39] Trautinger, F., Kindas-Mugge, I., Dekrout, B., Knobler, R. M., Metze, D., Expression of the 27-kDa heat shock protein in human epidermis and in epidermal neoplasms: an immunohistological study. Br J Dermatol 1995, 133, 194-202.
[40] Nagakubo, D., Taira, T., Kitaura, H., Ikeda, M., et al., DJ-1, a novel oncogene which transforms mouse NIH3T3 cells in cooperation with ras. Biochem Biophys Res Commun 1997, 231, 509-513.
[41] Le Naour, F., Misek, D. E., Krause, M. C., Deneux, L., et al., Proteomics-based identification of RS/DJ-1 as a novel circulating tumor antigen in breast cancer. Clin Cancer Res 2001, 7, 3328-3335.
[42] MacKeigan, J. P., Clements, C. M., Lich, J. D., Pope, R. M., et al., Proteomic profiling drug-induced apoptosis in non-small cell lung carcinoma: identification of RS/DJ-1 and RhoGDIalpha. Cancer Res 2003, 63, 6928-6934.
[43] Hod, Y., Differential control of apoptosis by DJ-1 in prostate benign and cancer cells. J Cell Biochem 2004, 92, 1221-1233.
[44] Davidson, B., Hadar, R., Schlossberg, A., Sternlicht, T., et al., Expression and clinical role of DJ-1, a negative regulator of PTEN, in ovarian carcinoma. Hum Pathol 2008, 39, 87-95.
[45] Kim, R. H., Peters, M., Jang, Y., Shi, W., et al., DJ-1, a novel regulator of the tumor suppressor PTEN. Cancer Cell 2005, 7, 263-273.
[46] Cheung, T. H., Lo, K. W., Yim, S. F., Chan, L. K., et al., Epigenetic and genetic alternation of PTEN in cervical neoplasm. Gynecol Oncol 2004, 93, 621-627.
[47] Lee, J. S., Choi, Y. D., Lee, J. H., Nam, J. H., et al., Expression of PTEN in the progression of cervical neoplasia and its relation to tumor behavior and angiogenesis in invasive squamous cell carcinoma. J Surg Oncol 2006, 93, 233-240.
[48] Morris, H. R., Genetics of Parkinson's disease. Annals of medicine 2005, 37, 86-96.
[49] Hong, T. M., Yang, P. C., Peck, K., Chen, J. J., et al., Profiling the downstream genes of tumor suppressor PTEN in lung cancer cells by complementary DNA microarray. Am J Respir Cell Mol Biol 2000, 23, 355-363.
[50] Nakano, T., Oka, K., Taniguchi, N., Manganese superoxide dismutase expression correlates with p53 status and local recurrence of cervical carcinoma treated with radiation therapy. Cancer Res 1996, 56, 2771-2775.
[51] Somiari, R. I., Sullivan, A., Russell, S., Somiari, S., et al., High-throughput proteomic analysis of human infiltrating ductal carcinoma of the breast. Proteomics 2003, 3, 1863-1873.
[52] Boasso, A., Herbeuval, J. P., Hardy, A. W., Winkler, C., Shearer, G. M., Regulation of indoleamine 2,3-dioxygenase and tryptophanyl-tRNA-synthetase by CTLA-4-Fc in human CD4+ T cells. Blood 2005, 105, 1574-1581.
[53] Gabay, C., Kushner, I., Acute-phase proteins and other systemic responses to inflammation. N Engl J Med 1999, 340, 448-454.
[54] Kozak, K. R., Su, F., Whitelegge, J. P., Faull, K., et al., Characterization of serum biomarkers for detection of early stage ovarian cancer. Proteomics 2005, 5, 4589-4596.
[55] Tanaka, S., Ramirez, F., The first intron of the human alpha2(I) collagen gene (COL1A2) contains a novel interferon-gamma responsive element. Matrix Biol 2007, 26, 185-189.

We claim:

1. A method to distinguish risk of progression of human papillomavirus (HPV) induced cancer comprising
a) determining the levels of one or more biomarkers selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cell or tissue sample;
b) comparing the levels of the one or more biomarkers in the cell or tissue sample to a control or reference level; and
c) identifying virally transformed cells based on the levels of the one or more biomarkers detected in the cells, wherein the presence of virally transformed cells is indicative of cancer.

2. The method of claim 1 wherein the cell or tissue sample is infected with HPV.

3. The method of claim 1 wherein cell or tissue sample is obtained from the cervix.

4. The method of claim 1 wherein the control is obtained from the same individual as the cell or tissue sample.

5. The method of claim 1, wherein the control is obtained from a different individual than from the cell or tissue sample.

6. The method of claim 1, wherein the cancer is cervical cancer.

7. The method of claim 1 wherein the cancer is cancer of the rectum, larynx, orthopharynx, nasopharynx, mouth, head and neck.

8. A method for detecting the transition of virally infected cells to virally transformed cells in cervical cancer comprising
a) determining the levels of one or more biomarkers for cervical cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cervical cell or cervical tissue sample;
b) comparing the levels of the one or more biomarkers for cervical cancer in the cervical cell or cervical tissue sample to a control or reference level; and
c) identifying virally transformed cervical cells based on the levels of the one or more biomarkers detected in the cells.

9. The method of claim 8 further comprising the step of reporting the presence or absence of virally transformed cervical cancer cells in the cervical cell or cervical tissue sample.

10. The method of claim 8 wherein the levels of the one or more biomarkers are detected using mass spectometry or immunocytochemistry.

11. The method of claim 8 wherein levels of cornulin, transthyretin, HSPB1, transferrin, Hspβ6 (HSP20), aflatoxin reductase, α2 type I collagen, cytokeratin 13, GST π, and cytokeratin 10 in the cell or cervical tissue sample that are lower than levels in noncancer cells are indicative of virally transformed cervical cancer cells.

12. The method of claim 8 wherein levels of PA28 β, DJ-1 protein, actin, Cl⁻ intracellular channel 1, cytokeratin 8, creatine kinase B, PA28 α in the cell or cervical tissue sample that are higher than levels in noncancer cells are indicative of virally transformed cervical cancer cells.

13. A method for distinguishing invasive cervical cancer cells, premalignant cervical cells, and noncancer cervical cells comprising
a) determining the levels of one or more biomarkers for cervical cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cervical cell or cervical tissue sample;
b) comparing the levels of the one or more biomarkers for cervical cancer in the cervical cell or cervical tissue sample to a control or reference level; and
c) identifying invasive cervical cancer cells, premalignant cervical cells, and noncancer cervical cells based on the levels of the one or more biomarkers detected in the cells.

14. The method of claim 13 further comprising the step of reporting the presence or absence of invasive cervical cancer cells in the cervical cell or cervical tissue sample.

15. The method of claim 13 wherein the levels of the one or more biomarkers are detected using mass spectometry or immunocytochemistry.

16. The method of claim 13 wherein levels of cornulin, transthyretin, HSPB1, transferrin, Hspβ6 (HSP20), aflatoxin reductase, a2 type I collagen, cytokeratin 13, GST π, and cytokeratin 10 in the cell or cervical tissue sample that are lower than levels in noncancer cells are indicative of invasive cervical cancer cells.

17. The method of claim 13 wherein levels of PA28β, DJ-1 protein, actin, Cl⁻ intracellular channel 1, cytokeratin 8, creatine kinase B, PA28 α in the cell or cervical tissue sample that are higher than levels in noncancer cells are indicative of invasive cervical cancer cells.

18. A method for monitoring the effects of a drug in the treatment of cervical cancer comprising
a) determining the levels of one or more biomarkers for cervical cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type 1 collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, trp-tRNA synthetase, and combinations thereof in a cervical cell or cervical tissue sample from subject before and after treatment with the drug; and
b) identifying changes in levels of the one or more biomarkers in the cervical cell or cervical tissue sample after treatment with the drug relative to before treatment of the drug wherein the changes in levels of the one or more biomarkers are indicative of effects of the treatment with the drug.

19. A method for selecting lead compounds for drug development for the treatment of cervical cancer comprising
a) contacting a test compound with one or more biomarkers for cervical cancer selected from the group consisting of cornulin, PA28 β, DJ-1, actin, transthyretin, HSPB1, Cl⁻ intracellular channel 1, cytokeratin 8, transferrin, Hspβ6 (HSP20), aflatoxin reductase, α2 type I collagen, creatine kinase B, cytokeratin 13, GST π, PA28 α, Manganese SOD, lamin A/C, serpin B1 (elastase inhibitor), serpin B3 (SCAA1), cytokeratin 10, cytokeratin 6A, and trp-tRNA synthetase;
b) assaying for binding of the test compound to one or more of the biomarkers; and
c) selecting the test compound that binds to one or more of the biomarkers for cervical cancer for drug development for the treatment of cervical cancer.

20. A method for detecting invasive cervical cancer cells comprising
a) obtaining a sample of cervical tissue from a subject;
b) quantifying levels of cornulin in the sample of cervical tissue;
c) comparing the levels of cornulin in the sample of cervical tissue to levels of cornulin in noncancer cells, wherein decreased levels of cornulin in the sample of cervical tissue relative to levels of cornulin in noncancer cells is indicative of invasive cervical cancer cells.

21. A method for distinguish premalignant cells from invasive cervical cancer cells comprising
a) obtaining a sample of cervical tissue from a subject;
b) quantifying levels of trp-tRNA synthetase in the sample of cervical tissue;
c) comparing the levels of trp-tRNA synthetase in the sample of cervical tissue to levels of trp-tRNA synthetase in noncancer cells, wherein elevated levels of trp-tRNA synthetase in the sample of cervical tissue relative to a reference level of trp-tRNA in premalignant cells is indicative of invasive cervical cancer cells.
